(19) United States
(12) Patent Application Publication
(10) Pub. No.: US 2008/0269724 A1
(43) Pub. Date: Oct. 30, 2008

(54) Implantable drug delivery device with programmable rate capacitor charge control

(75) Inventors: Scott A. Sarkinen, Greenfield, MN (US); James M. Haase, Maplewood, MN (US); Ronald L. Mezera, Lake Elmo, MN (US); Christian Peclat, Neuchatal (CH)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Medtronic, Inc., Minneapolis, MN (US)

(21) Appl. No.: 11/796,604

(22) Filed: Apr. 27, 2007

(51) Int. Cl. A61M 5/142

(52) U.S. Cl. 604/891.1

(57) ABSTRACT

An implantable drug delivery device includes a pump motor that is asserted by drive currents from a storage capacitor. A programmable rate charge control delivers charging current from a battery to the storage capacitor based upon a programmable charge rate value, a minimum battery voltage value, sensed charging current, and sensed battery voltage. When sensed battery voltage droops to below a threshold value, the charge control reduces the charging rate value until other electrical loads within the drug device have been serviced and battery voltage is restored. The charge control also monitors capacitor voltage and provides a charge complete signal to a motor control, which then connects the pump motor to the storage capacitor to produce a pump stroke. Efficiency of charging is enhanced by controlling the charging at a programmable substantially constant rate.

### BACKGROUND

The present invention relates to implantable medical devices. In particular, the present invention relates to a charge control for controlling charging of a capacitor from a battery and subsequently delivering stored energy from the capacitor to a pump motor.

Implantable drug delivery devices are used to provide patients with long-term dosage or infusion of a drug or other therapeutic agent. Implantable drug delivery devices may be categorized as either passive or active devices.

Passive drug delivery devices typically rely upon a pressurized drug reservoir to deliver the drug. The reservoir may be filled using a syringe. The drug is then delivered to the patient using force provided by the pressurized reservoir.

Active drug delivery devices include a pump or metering system to deliver the drug into the patient's system. The pump is electrically powered to deliver the drug from a reservoir through a catheter to a selected location within the patient's body. The pump typically includes a battery as its power source for both the pump and for the electronic circuitry used to control flow rate of the pump and to communicate through telemetry to an external device to allow programming of the pump.

Battery life is an important consideration for all implantable medical devices. With an implantable drug delivery device, efficiency of the driver circuitry that powers the pump motor is an important consideration. In one type of driver configuration, the pump motor is driven from electrical energy stored by a storage capacitor. The capacitor serves as a low-impedance, short-term energy reservoir to deliver sufficient power to the pump motor during assertion. During pump operation, the motor will be asserted periodically for a short period of time to provide a pulse flow of the drug, and followed by a longer period until the next assertion.

The efficiency of the driver circuitry can have an important effect on the lifetime of the battery, overall volume of the device (including battery size, capacitor size, and size of the circuitry required), and on the overall cost of the device. Considerations in the overall efficiency of the driver include the efficiency of charging the storage capacitor, and the efficiency of delivering energy stored in the storage capacitor to the pump motor.

### SUMMARY

An implantable drug delivery device includes a pump motor, a battery, and a driver powered by the battery for operating the motor. The driver includes a storage capacitor for storing electrical energy from the battery, a charge control for charging the storage capacitor, and a motor control for delivering the electrical energy from the storage capacitor to the pump motor. The charge control delivers charging current from the battery to the capacitor based upon a charging rate value, a minimum battery voltage value, sensed charging current, and sensed battery voltage.

### BRIEF DESCRIPTION OF THE DRAWINGS

FIG. 1 is a block diagram showing an implantable drug delivery device.

FIG. 2 is a schematic diagram showing the battery, charge control, storage capacitor, motor control, and motor of one embodiment of the device of FIG. 1.

FIG. 3 is a schematic diagram of one embodiment of the monitor of the device of FIG. 1.

### DETAILED DESCRIPTION

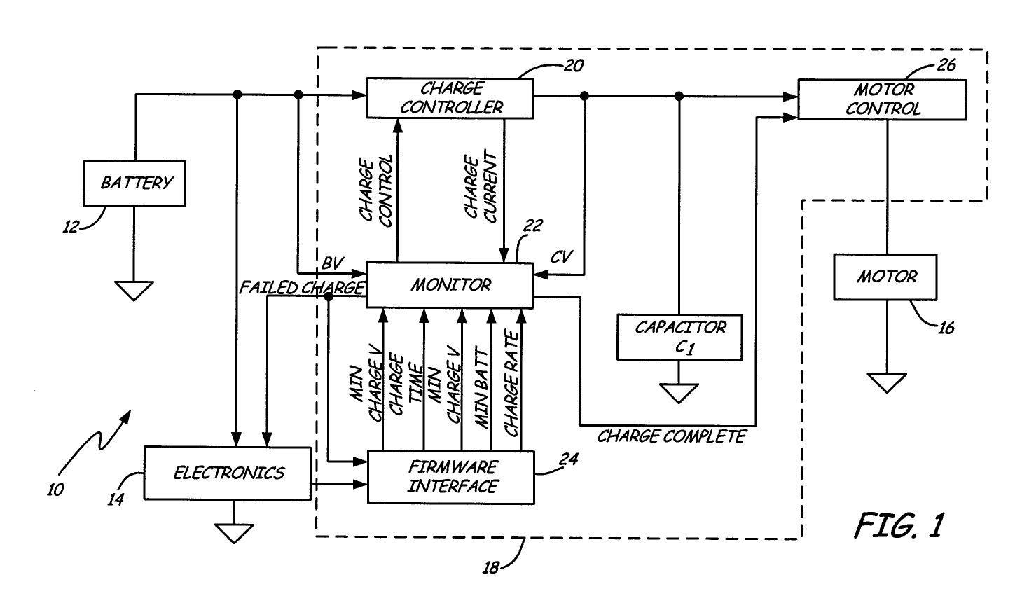

FIG. 1 shows implantable drug delivery device 10, which includes battery 12, device electronics 14, motor 16, and electronic motor driver 18 (which includes charge controller 20, monitor 22, firmware interface 24, storage capacitor Cl, and motor control 26).

Battery 12 acts as a power source that provides all of the electrical energy for operation of implantable drug delivery device 10. In particular, battery 12 provides the electrical energy to power device electronics 14, as well as the power used by motor driver 18 to generate electrical pulses delivered to motor 16 to pump a drug or other therapeutic agent to a desired location within the patient's body. Battery 12 can make use of any battery technology consistent with the lifetime, physical size, and performance requirements for an implantable battery. The battery technologies can include, for example, CSVO cathode technology that delivers medium capacity and high pulse current during operation. Another alternative is hybrid cathode technology that features high energy density but also has high source resistance.

Device electronics 14 typically include a microprocessor or other programmable digital electronics, together with associated memory and timing circuitry for controlling and coordinating the operation of device 10. Device electronics 14 may also include an antenna and transceiver for RF telemetry, to allow communication with an external device, so that drug delivery device 10 can be programmed to deliver a drug at a selected rate.

Motor 16 is, in one embodiment, a solenoid type pump. When the motor is asserted, a solenoid coil is energized, which produces an electromagnetic field causing a solenoid plunger or actuator to move. Motor 16 may also include a spring bias, which returns the actuator to its original position when the solenoid coil is no longer energized. Motor 16 typically is asserted or energized for a relatively short time period, with a relatively long period between successive assertions. The delivery rate of the pump will depend on the period of time between successive assertions of the motor that produce a pump stroke. Assertion time of motor 16 may be on the order of milliseconds (e.g. 5 milliseconds) and the period between motor assertion will vary with delivery rate and may be on the order of several seconds (e.g. 3 seconds).

Motor driver 18 isolates motor 16 from battery 12 through charge controller 20 and motor control 26. Motor 16 is driven by energy stored in storage capacitor Cl, rather than directly from battery 12. As a result, a low impedance load presented by motor 16 is not directly connected to battery 12, and therefore does not cause a decrease or droop in battery voltage each time a motor assertion occurs. The stability of the battery voltage is important to proper functioning of device electronics 16, as well as the electrical devices of driver 18.

Power delivered by motor control 26 to motor 16 is provided from storage capacitor C₁. Charge controller 20, in conjunction with monitor 22 and firmware interface 24, controls the charging of storage capacitor C₁ to enhance charging efficiency. Charge controller 20 delivers a programmable substantially constant charging current to storage capacitor C₁ during each charging operation. This provides improved efficiency, because storage capacitor C₁, when it begins charging, is capable of accepting a large amount of current, while providing a very slow increase in voltage. A high charging current during initial charging results in additional energy loss in the internal resistance of battery 12. By maintaining charging current at a substantially constant level throughout the charging operation, less energy loss occurs in battery 12, and the charging efficiency is improved.

Monitor 22 receives inputs representing sensed charge current from charge controller 20, sensed battery voltage BV, and sensed capacitor voltage CV. Monitor 22 provides charge controller 20 with a Charge Control signal that controls operation of the switches within charge controller 20. The Charge Control signal is a function of sensed battery voltage BV, charge current, a programmable charge rate value and a programmable minimum battery voltage value (provided to monitor 22 by firmware interface 24). Monitor 22 controls the Charge Control signal so that the charge current will be maintained at or near the charge rate value. If battery voltage BV begins to droop, for example as a result of operation of device electronics 14, monitor 22 will modify the Charge Control signal to reduce or even stop charging until the current draw from device electronics 14 is reduced and battery voltage BV increases above the minimum battery voltage value. In one embodiment, as the battery voltage BV increases, monitor 22 will vary the Charge Control signal to gradually increase the charge current until it is restored to the programmable charge rate value provided by firmware interface 24.

The coordination of the power demands of motor driver 18 with the demands of other loads operated by device electronics 14 prevents battery voltage droop that may adversely effect operation of device electronics 14. It also enhances efficiency of charging by curtailing or reducing the charging operation when battery voltage is low.

Monitor 22 also controls the discharging of storage capacitor Cl by motor control 28. Monitor 22 receives a minimum charge voltage value for storage capacitor Cl, a maximum charge value for storage capacitor Cl, and a charge time (which is the time period between motor assertions, and determines pump delivery rate). All three values are programmable through device electronics 14 and firmware interface 24. In other words, all of the programmable values provided to monitor 22 can be changed, as desired, by downloading new values via telemetry to device electronics 14, which then provides those values to firmware interface 24.

Monitor 22 uses the sensed battery voltage BV and capacitor voltage CV to determine when capacitor 26 is charged sufficiently so that motor control 26 can assert motor 16 by delivering electrical energy from storage capacitor C₁ to motor 16.

Monitor 22 determines when capacitor voltage CV has reached the minimum charge value, which is provided by firmware interface 24. Monitor 22 continues to monitor voltage CV to determine whether a maximum charge voltage is reached. The maximum charge voltage is a programmable percentage of the sensed battery voltage.

If capacitor voltage CV reaches the maximum charge voltage before the charge time has expired, monitor 22 provides a Charge Complete signal to motor control 26. In response to the Charge Complete signal, motor control 26 causes current from storage capacitor C₁ to be delivered to motor 16 for a time period tₒₙ sufficient to produce a full stroke of the solenoid pump.

If the charge time expires before a maximum charge voltage has been achieved by storage capacitor C₁, but the minimum charge voltage was reached, then monitor 22 still produces the Charge Complete signal. In other words, even though a maximum charge not achieved on storage capacitor C₁, motor 16 will again be asserted as long as there is at least the minimum charge on storage capacitor C₁.

If the charge time interval expires without the capacitor voltage CV reaching the minimum charge value, then monitor 22 provides a Failed Charge signal to both device electronics 14 and firmware interface 24. The Failed Charge signal may represent only a temporary condition, or may signal a longer term problem affecting operation of implantable drug delivery device 10. Device electronics 14 can provide a signal via telemetry to an external device to indicate that a failed charge condition has occurred.

The Failed Charge signal can also be used to modify the programmed values (or select alternative values) that are provided by firmware interface 24 to monitor 22. A change in values may result in the next operating cycle successfully charging storage capacitor C₁ to at least the minimum charge voltage. For example, in response to a Failed Charge signal, the charge rate may be modified to increase the charge current delivered by charge controller 20 to storage capacitor C₁.

Firmware interface 24 allows the programmed values or set points used by monitor 22 to be changed to offer different modes of operation. For example, during initial setup of drug delivery device 10, prior to the implantation, device 10 may be filled with a fill fluid such as water that must be removed so that device 10 can be filled with the drug. By providing a command to device electronics 14 by telemetry, a fast operating mode can be initiated to accelerate the pumping of the fill fluid in preparation for being filled with a drug. This can be done by changing the charge time, which changes the rate at which motor 16 is asserted. Other set points, such as the charge rate, also may be changed in order to accelerate charging of storage capacitor C₁ to accommodate a higher pump rate.

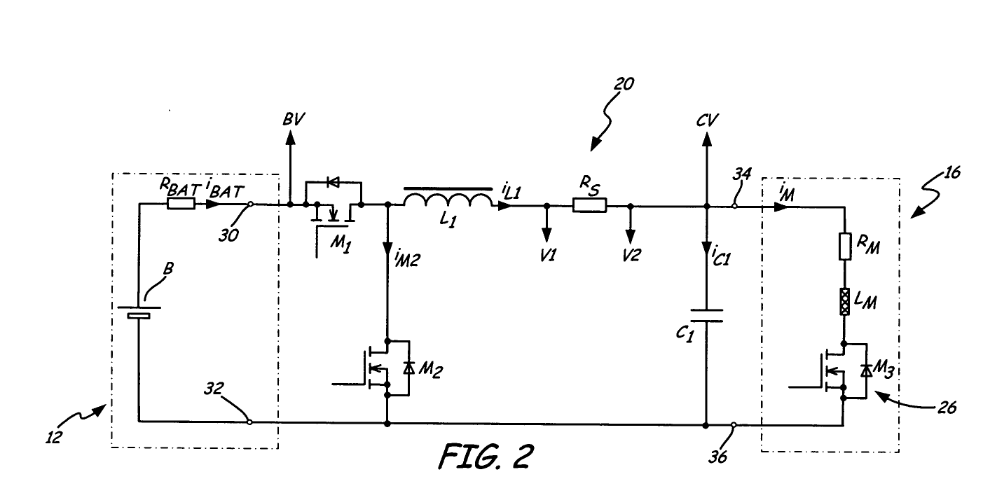

FIG. 2 is a schematic diagram illustrating battery 12, motor 16, charge controller 20, storage capacitor C₁ and motor control 26 in one embodiment of the invention. Battery 12 is shown as an ideal battery B and internal resistance R_{BAT} between battery terminals 30 and 32. Motor 16 is connected between motor terminals 34 and 36 and represents a load having a real component R_{M} and an inductive component L_{M}. Storage capacitor C₁, is connected across motor terminals 34 and 36.

Charge controller 20 includes electronic switches M₁ and M₂, inductor L₁ and sense resistor R_{S}. Switches M₁ and M₂ of charge controller 20 are operated by the Charge Control signal delivered by monitor 22. Switches M₁ and M₂ are operated simultaneously so that one switch is on while the other is off.

When switch M₁ is on, current i_{BAT} from battery 12 flows through M₁, inductor L₁, and sense resistor R_{S} to storage capacitor C₁. Switch M₂ is turned off, as is switch M₃ of motor control 26. As a result, all of the battery current i_{BAT} flows through switch M₁ and inductor L₁, and then through sense resistor R_{S} to capacitor C₁. Thus, i_{BAT} equals i_{L1} equals i_{C1}.

When the current flowing through sense resistor R_{S} reaches the charge rate set point, as indicated by the difference between voltage V₁ and voltage V2, monitor 22 changes the Charge Control signal so that M₁ is turned off and M₂ is turned on. The current flowing in resistor L₁ at the time that M₁ and M₂ change state represents stored energy that otherwise could be lost. By providing a current path through transistor M₂, a charging circuit is maintained which allows the energy stored in inductor L₁ to be transferred to storage capacitor C₁. When the current through sense resistor R_{S} diminishes, monitor 22 again reverses switches M₁ and M₂ so that current again can flow through M₁, L₁ and R_{S} due to storage capacitor C₁. The active transfer circuit formed by switch M₁, switch M₂, and inductor L₁, in conjunction with the current sensing provided by resistor R_{S}, provides high efficiency charging of storage capacitor C₁ from battery 12. The charging current is maintained substantially constant at a level set by the charge rate value provided by firmware interface 24 to monitor 22. This increases the efficiency of charging by not permitting extremely high currents, and thus high losses in battery 12, when charging of storage capacitor C₁ first begins following a motor assertion.

In the embodiment shown in FIG. 2, motor control 26 is shown as a single electronic switch M₃ connected in series with components R_{M} and L_{M} of motor 16 between terminals 34 and 36. In other embodiments, motor control 26 may include multiple electronic switches connected in a control circuit with motor 16.

Once storage capacitor C₁ has been charged and monitor 22 produces a Charge Complete signal, switch M₃ of motor control 26 is turned on. This establishes a current path from storage capacitor C₁ through terminal 34, motor components R_{M} and L_{M}, and switch M₃ to terminal 36. During the discharge of storage capacitor C₁ to motor 16, switch M₁ of charge controller 20 is turned off, so that battery 12 is isolated from motor 16. The charging cycle begins again after motor assertion is complete and switch M₃ is again turned off.

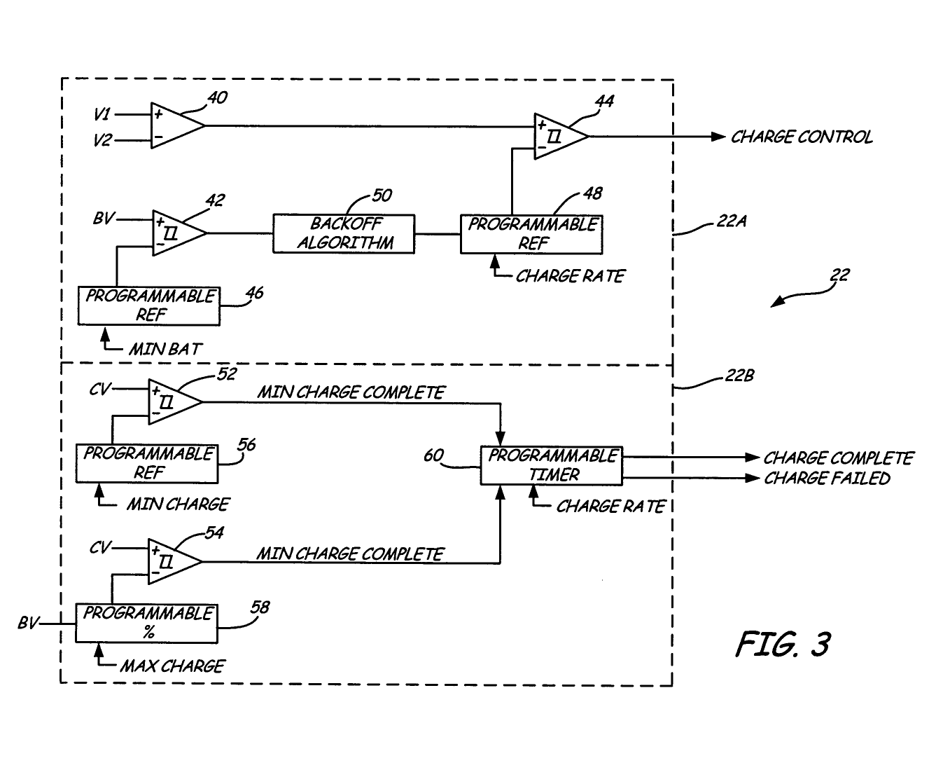

FIG. 3 is a schematic diagram illustrating one embodiment of monitor 22. In this embodiment, monitor 22 includes two major sections 22A and 22B. Section 22A produces the Charge Control signal based upon the charge current sense voltages V₁ and V₂, battery voltage BV, and the minimum battery voltage and charge rate set point values from firmware interface 24. Section 22B produces the Charge Complete and Charge Failed signals based upon capacitor voltage CV, battery voltage BV, and the minimum charge, maximum charge and charge time set point values from firmware interface 24.

Monitor section 22A includes differential amplifiers 40 and 42, comparator 44, programmable references 46 and 48, and backoff algorithm 50. Voltages V₁ and V₂ represent voltages measured on opposite sides of current sense resistance R_{S} in FIG. 2. The difference between voltage V₁ and V₂ is a function of the charge current flowing through resistor R_{S}. Amplifier 40 provides an output to the noninverting input of comparator 44 representing the difference V1-V2, which represents current i_{L1} shown in FIG. 2 (since i_{L1}=(V₁-V₂)/R_{S}).

Amplifier 42 compares battery voltage BV with a programmable reference value produced by programmable reference 46 in response to the minimum battery value from firmware interface 24. The output of amplifier 42 is provided to backoff algorithm 50, which provides an input to programmable reference 48 that is used in conjunction with the charge rate set point to provide a reference level to the inverting input of comparator 44. The reference level can range from zero up to maximum level representing the maximum current defined by the charge rate set point. When battery voltage droops to below the minimum battery level, backoff algorithm 50 will cause the reference level to comparator 44 to be decreased. This decrease may be all the way to zero, or to some predefined percentage of the charge rate set point. As battery voltage then increases above the minimum battery voltage, backoff algorithm 50 provides an input that causes programmable reference 48 to vary the reference level until it reaches a maximum defined by the charge rate set point.

The output of comparator 44 is the Charge Control signal controls the state of switches M₁ and M₂ in FIG. 2. The Charge Control signal may be generated as complimentary signals by also inverting the output of comparator 44, so that switch M₁ gets one of the complementary signals and switch M₂ gets the other signal.

Monitor section 22B monitors capacitor voltage CV and battery voltage BV to determine when charging of storage capacitor C₁ has been successful and is complete. Monitor section 22B includes comparators 52 and 54, programmable references 56 and 58, and programmable timer 60. Comparator 52, in conjunction with programmable reference 56, determines when a minimum charge of storage capacitor C₁ has been completed. Comparator 52 compares capacitor voltage CV with a minimum charge level produced by programmable reference 56 in response to the minimum charge set point from firmware interface 24. When capacitor voltage CV exceeds the minimum charge level, a Minimum Charge Complete signal is supplied by comparator 52 to programmable timer 60.

Comparator 54 and programmable reference 58 determine when a maximum charge has been achieved. Programmable reference 58 produces a maximum charge level based upon the sensed battery voltage BV and a maximum charge percentage set point received from firmware interface 24. Comparator 54 compares the sensed capacitor voltage CV with the maximum charge level, which is a percentage of the sensed battery voltage BV. When capacitor voltage CV exceeds the maximum charge level, a Maximum Charge Complete signal is supplied to programmable timer 60.

Programmable timer 60 defines a charge time or time interval that represents the time between successive assertions of motor 16. This charge time, therefore, defines the pump delivery rate of implantable drug delivery device 10.

Each time a Charge Complete or Charge Failed signal is produced by programmable timer 60, it resets and begins a new charge time period. The length of the charge time period is based upon a charge time set point received from firmware interface 24. If programmable timer 60 receives a Maximum Charge Complete signal before the time charge interval expires, it generates a Charge Complete signal. It will also produce a Charge Complete signal if the Minimum Charge Complete signal has been received by the time that the charge time interval has expired. In either case, the Charge Complete signal allows motor control 26 to assert motor 16. If the charge time interval times out without the minimum charge complete signal having been generated, programmable timer 60 produces a Charge Failed signal.

The motor driver of the present invention provides a more efficient, programmable charging of a storage capacitor, which is then used to deliver pulses to operate a pump motor. The motor driver provides isolation between the battery and the motor, and coordinates the charging of the capacitor with other loads presented to the battery by the electronics of the implantable drug delivery device.

Although specific circuits have been illustrated, other implementations of the invention may use different components, circuits and technologies. For example, FIG. 2 shows an implementation using discrete electrical components, but the functions of charge controller 20 and motor control 26 can also be implemented in an application specific integrated circuit (ASIC). Other portions of the device, as shown in FIGS. 1 and 3 could also be included in an ASIC. Although FIG. 3 shows an analog circuitry implementation of monitor 22, some or all of the functions can be implemented using digital circuitry. Although control of the charging current at a programmable substantially constant rate has been described using switching circuitry, the control can also be implemented using transistors, amplifiers and other circuits to maintain charging current constant.

Although the present invention has been described with reference to preferred embodiments, workers skilled in the art will recognize that changes may be made in form and detail without departing from the spirit and scope of the invention.

1. An implantable medical device comprising:
an electromagnetic pump having a coil that can be energized to produce a pump stroke;
a power source;
a storage capacitor for storing electrical energy from the power source and for delivering stored electrical energy to the coil; and
a charge control system for controlling charging of the storage capacitor as a function of sensed power source voltage, and sensed charging current.

2. The device of claim 1, wherein the charge control system decreases charging current to the storage capacitor when sensed power source voltage is below a minimum power source voltage level.

3. The device of claim 2, wherein the charge control system increases charging current to the storage capacitor following an increase in sensed power source voltage to a level above the minimum power source voltage level.

4. The device of claim 1, wherein the charge control system includes a charge controller circuit for supplying charging current from the power source to the storage capacitors.

5. The device of claim 4, wherein the charge control system controls charging of the storage capacitor at a programmable substantially constant rate.

6. The device of claim 5, wherein the charge control system includes a monitor for controlling operation of the charge controller circuit based upon the sensed charging current and a programmable charge rate value representing a desired charging current level.

7. The device of claim 6, wherein the monitor causes the charge controller circuit to change current flow from the power source to the storage capacitor when the sensed charging current varies from the desired charging current level.

8. The device of claim 4, wherein the charge controller circuit includes a purality of switches from switching current flowing between the power source and the storage capacitor, and an inductor in a current path between the power source and the storage capacitor.

9. The device of claim 1, wherein the charge control system controls charging current flow to the storage capacitor at a substantial constant current level.

10. The device of claim 1, wherein the charge control system determines when charging of the storage capacitor is complete.

11. The device of claim 10, wherein the charge control system determines whether storage capacitor voltage has attained a minimum charge level and a maximum charge level.

12. The device of claim 11, wherein the charge control system provides a Charge Complete signal if either the maximum charge level is attained, or the minimum charge level is attained before an end of a charging interval.

13. The device of claim 12, wherein the charge control system provides a Charge Failed signal if the minimum charge level has not been attained by the end of the charging interval.

14. The device of claim 11, wherein the minimum charge level is a programmable value.

15. The device of claim 11, wherein the maximum charge level is a programmable percentage of sensed power source voltage.

16. The device of claim 11 and further comprising:
a motor control for controlling delivery of stored electrical energy from the storage capacitor to the coil.

17. The device of claim 16, wherein the motor control causes stored electrical energy from the storage capacitor to be delivered to the coil in response to a determination by the charge control system that charging of the storage capacitor is complete.

18. The device of claim 1, wherein the charge control system comprises:
a charge controller for regulating flow of charging current to the storage capacitor;
a firmware interface for providing programmable set point values; and
a monitor for producing control signals to the charge controller based upon sensed power source voltage, sensed charging current, and programmable set point values.

19. The device of claim 18 and further comprising:
a motor control for controlling delivery of stored electrical energy to the coil.

20. The device of claim 19, wherein the monitor provides a Charge Complete signal to the motor control to indicate that the storage capacitor is ready for delivery of stored electrical energy to the coil.

21. The device of claim 20, wherein the monitor provides the Charge Complete signal based upon sensed power source voltage, sensed storage capacitor voltage, and programmable set point values.

22. An implantable drug delivery device comprising:
a battery;
a storage capacitor;
a pump motor;
a charge control system for charging the storage capacitor from the battery at a substantially constant programmable rate with a charging current; and
a motor control circuit for delivering electrical energy stored in the storage capacitor to the pump motor to produce a pump stroke.

23. The device of claim 22, wherein the charge control system controls the charging current as a function of a desired charging current level based on the programmable rate and a signal representative of sensed charging current.

24. The device of claim 22, wherein the charge control system circuit varies the desired charging current level as a function of sensed battery voltage.

25. The device of claim 24, wherein the charge control system decreases charging current to the storage capacitor when sensed battery voltage is below a minimum battery voltage level.

26. The device of claim 25, wherein the charge control system increases charging current to the storage capacitor following an increase in sensed battery voltage to a level above the minimum battery voltage level.

27. The device of claim 22, wherein the charge control system includes a plurality of switches for switching current flowing between the power source and the storage capacitor.

28. The device of claim 27, wherein the charge control system controls charging based upon the sensed charging current and a charge rate value representing a desired charging current level.

29. The device of claim 22, wherein the charge control system interrupts current flow from the battery to the storage capacitor when the sensed charging current exceeds the desired charging current level.

30. The device of claim 22, wherein the charge control system determines when charging of the storage capacitor is complete.

31. The device of claim 30, wherein the charge control system determines whether storage capacitor voltage has attained a minimum charge level and a maximum charge level.

32. The device of claim 31, wherein the charge control system provides a Charge Complete signal if either the maximum charge level is attained, or the minimum charge level is attained before an end of a charging interval.

33. The device of claim 32, wherein the charge control system provides a Charge Failed signal if the minimum charge level has not been attained by the end of the charging interval.

34. The device of claim 31, wherein the minimum charge level is a programmable value.

35. The device of claim 31, wherein the maximum charge level is a programmable percentage of sensed power source voltage.

36. The device of claim 30, wherein the motor control circuit causes stored electrical energy from the storage capacitor to be delivered to the pump motor in response to a determination by the charge control system that charging of the storage capacitor is complete.

37. The device of claim 22, wherein the charge control system comprises:
a charge controller for regulating flow of charging current tot the storage capacitor;
a firmware interface for providing programmable set point values; and
a monitor for producing control signals to the charge controller based upon sensed battery voltage, sensed charging current, and programmable set point values.

38. The device of claim 22, wherein the charge control system provides a Charge Complete signal to the motor control to indicate that the storage capacitor is ready for delivery of stored electrical energy to the pump motor.

39. The device of claim 36, wherein the charge control system provides the Charge Complete signal based upon sensed power source voltage, sensed storage capacitor voltage, and programmable set point values.

40. An implantable drug delivery device comprising:
a battery;
an electromechanical pump motor;
a motor driver including a storage capacitor, the motor driver charging the storage capacitor from the battery at a programmable substantially constant rate with a charging current and driving the pump motor with electrical energy from the storage capacitor.

41. The device of claim 40, wherein the motor driver decreases charging current to the storage capacitor when sensed battery voltage is below a minimum power source voltage level, and increases charging current to the storage capacitor following an increase in sensed battery voltage to a level above the minimum battery voltage level.

42. The device of claim 40, wherein the motor driver controls the charging current based upon a desired charging current level based on the programmable rate and a signal representative of sensed charging current.

43. The device of claim 40, wherein the motor driver determines when charging of the storage capacitor is complete based upon sensed storage capacitor voltage.

44. The device of claim 43, wherein the motor driver determines whether storage capacitor voltage has attained a minimum charge level and a maximum charge level.

45. The device of claim 44, wherein the motor driver provides a Charge Complete signal if either the maximum charge level is attained, or the minimum charge level is attained before an end of a charging interval.

46. The device of claim 45, wherein the motor driver provides a Charge Failed signal if the minimum charge level has not been attained by the end of the charging interval.

47. A method of operating a drug delivery device, the method comprising:
charging a storage capacitor from a battery at a programmable substantially constant rate with a charging current; and
delivering stored electrical energy from the storage capacitor to a pump motor.

48. The method of claim 47 and further comprising:
decreasing charging current to the-storage capacitor when sensed battery voltage is below a minimum power source voltage level.

49. The method of claim 48 and further comprising:
increasing charging current to the storage capacitor following an increase in sensed battery voltage to a level above the minimum battery voltage level.

50. The method of claim 47 and further comprising:
determining when charging of the storage capacitor is complete based upon sensed storage capacitor voltage.

51. The method of claim 50 and further comprising:
determining when charging of the storage capacitor is complete includes determining whether storage capacitor voltage has attained a minimum charge level and a maximum charge level.

52. The method of claim 51 and further comprising:
providing a Charge Complete signal if either the maximum charge level is attained, or the minimum charge level is attained before an end of a charging interval.

53. The method of claim 52 and further comprising:
providing a Charge Failed signal if the minimum charge level has not been attained by the end of the charging interval.

54. The method of claim 50, wherein delivering stored electrical energy to the pump motor occurs after determining that charging of the storage capacitor is complete.

55. The method of claim 47, wherein charging the storage capacitor includes operating a plurality of switches to switch current flowing between the power source and the storage capacitor.

56. The method of claim 47, wherein charging the storage capacitor is based upon a sensed charging current and a desired charging current level based on the programmable rate.

57. The method of claim 45, wherein charging the storage capacitor includes changing current flow from the battery to the storage capacitor when the sensed charging current varies from a desired charging current level.
